(19) United States
(12) Patent Application Publication
(10) Pub. No.: US 2018/0140223 A1
(43) Pub. Date: May 24, 2018

(54) CALIBRATION APPARATUS FOR A MEDICAL TOOL

(71) Applicants: Leila KHERADPIR, Toronto (CA); William LAU, Toronto (CA); Gal SELA, Toronto (CA); Neil WITCOMB, Toronto (CA)

(72) Inventors: Leila KHERADPIR, Toronto (CA); William LAU, Toronto (CA); Gal SELA, Toronto (CA); Neil WITCOMB, Toronto (CA)

(21) Appl. No.: 15/315,945

(22) PCT Filed: Oct. 17, 2014
(86) PCT No.: PCT/CA2014/051004

§ 371 Date: Dec. 2, 2016

(51) Int. Cl. A61B 5/06; A61B 34/20

(52) U.S. Cl. CPC A61B 5/064; A61B 5/6848; A61B 34/20

(57) ABSTRACT

A calibration apparatus is provided for calibrating a medical tool having a tool tracking marker. The medical tool and the calibration apparatus are for use with a medical navigation system. The calibration apparatus comprises a frame, a frame tracking marker attached to the frame, and a reference point formed on the frame. The reference point provides a known spatial reference point relative to the frame tracking marker.

### TECHNICAL FIELD

The present disclosure is generally related to image guided medical procedures, and more specifically to a calibration apparatus for a medical tool.

### BACKGROUND

The present disclosure is generally related to image guided medical procedures using a surgical instrument, such as a fibre optic scope, an optical coherence tomography (OCT) probe, a micro ultrasound transducer, an electronic sensor or stimulator, or an access port based surgery.

In the example of a port-based surgery, a surgeon or robotic surgical system may perform a surgical procedure involving tumor resection in which the residual tumor remaining after is minimized, while also minimizing the trauma to the intact white and grey matter of the brain. In such procedures, trauma may occur, for example, due to contact with the access port, stress to the brain matter, unintentional impact with surgical devices, and/or accidental resection of healthy tissue. A key to minimizing trauma is ensuring that the spatial reference of the patient and the medical tools used in the procedure as understood by the surgical system is as accurate as possible.

FIG. 1 illustrates the insertion of an access port into a human brain, for providing access to internal brain tissue during a medical procedure. In FIG. 1, access port 12 is inserted into a human brain 10, providing access to internal brain tissue. Access port 12 may include such instruments as catheters, surgical probes, or cylindrical ports such as the NICO BrainPath. Surgical tools and instruments may then be inserted within the lumen of the access port in order to perform surgical, diagnostic or therapeutic procedures, such as resecting tumors as necessary. The present disclosure applies equally well to catheters, DBS needles, a biopsy procedure, and also to biopsies and/or catheters in other medical procedures performed on other parts of the body.

In the example of a port-based surgery, a straight or linear access port 12 is typically guided down a sulci path of the brain. Surgical instruments would then be inserted down the access port 12.

Optical tracking systems, used in the medical procedure, track the position of a part of the instrument that is within line-of-site of the optical tracking camera. These optical tracking systems require a knowledge of the dimensions of the instrument being tracked so that, for example, the optical tracking system knows the position in space of a tip of a medical instrument relative to the tracking markers being tracked.

Conventional systems have shortcomings with respect to establishing and maintaining the reference between the tracking markers located on a medical instrument and the point of interest on the instrument relative to those tracking markers because instruments can bend or deform over time. Therefore, there is a need for an improved calibration of optical tracking systems with respect to the medical instruments that those tracking systems track.

### SUMMARY

One aspect of the present disclosure provides a calibration apparatus for calibrating a medical tool having a tool tracking marker. The medical tool and the calibration apparatus are for use with a medical navigation system. The calibration apparatus comprises a frame, a frame tracking marker attached to the frame, and a reference point formed on the frame. The reference point provides a known spatial reference point relative to the frame tracking marker.

The frame tracking marker may include at least one of a passive reflective tracking sphere, an active infrared (IR) marker, an active light emitting diode (LED), and a graphical pattern. The frame may haves at least three tracking markers attached to a same side of the frame. The reference point may include a divot and the medical tool has at least three tracking markers attached thereto and a tip of the medical tool may be insertable into the divot to abut against a floor of the divot for validation of the medical tool dimensions by the medical navigation system.

Another aspect of the present disclosure provides a medical navigation system having a medical tool, a calibration apparatus, and a controller. The medical tool has a tool tracking marker. The calibration apparatus is for calibrating the medical tool and the calibration apparatus has a frame, a frame tracking marker attached to the frame, and a reference point formed on the frame. The reference point provides a known spatial reference point relative to the frame tracking marker. The controller has a sensor coupled to the controller for detecting the tracking makers. The sensor provides a signal to the controller indicating positions of the tracking markers in space.

Another aspect of the present disclosure provides a method of verifying dimensions of a medical tool having an attached tool tracking maker using a calibration apparatus having a frame, a frame tracking marker attached to the frame and a reference point formed on the frame. The reference point provides a known spatial reference point relative to the frame tracking marker. The method comprises detecting the tool tracking maker and the frame tracking maker; calculating the expected spatial relationship of the tool tracking maker relative to the frame tracking maker; and reregistering the tool when the dimensions of the medical tool have changed beyond a threshold.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the insertion of an access port into a human brain, for providing access to internal brain tissue during a medical procedure;

FIG. 2 shows an exemplary navigation system to support minimally invasive surgery;

FIG. 3 is a block diagram illustrating a control and processing system that may be used in the navigation system shown in FIG. 2;

FIG. 4A is a flow chart illustrating a method involved in a surgical procedure using the navigation system of FIG. 2;

FIG. 4B is a flow chart illustrating a method of registering a patient for a surgical procedure as outlined in FIG. 4A;

FIG. 5 is a perspective drawing illustrating an exemplary tracked instrument with which aspects of the present application may be applied; and

FIG. 6 is a perspective drawing illustrating the tracked instrument shown in FIG. 5 inserted into a calibration apparatus;

FIG. 7 is perspective drawing illustrating in isolation the calibration apparatus shown in FIG. 6;

FIG. 8 is a front view of the calibration apparatus shown in FIG. 7;

FIG. 9 is a rear view of the calibration apparatus shown in FIG. 7;

FIG. 10 is a right side view of the calibration apparatus shown in FIG. 7;

FIG. 11 is a left side view of the calibration apparatus shown in FIG. 7;

FIG. 12 is a top view of the calibration apparatus shown in FIG. 7;

FIG. 13 is bottom view of the calibration apparatus shown in FIG. 7; and

FIG. 14 is a flow chart illustrating a method for verifying and reregistering a medical tool.

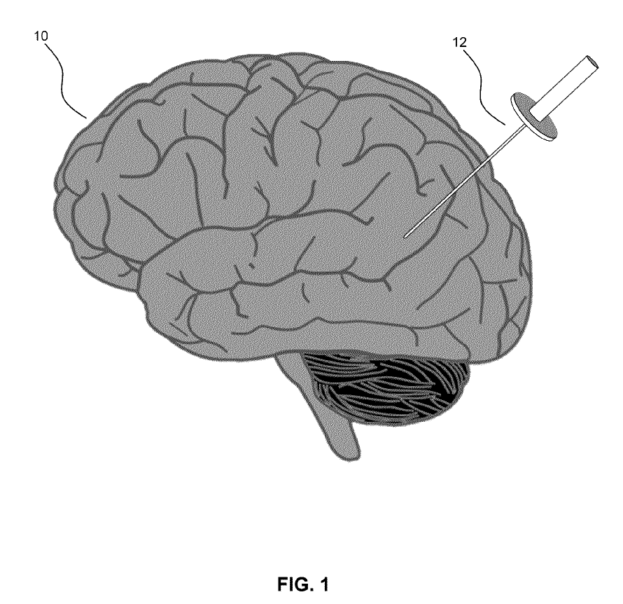

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein the terms “comprises” and “comprising” are to be construed as being inclusive and open ended, and not exclusive. Specifically, when used in the specification and claims the terms “comprises” and “comprising” and variations thereof mean the specified features steps or components are included. These terms are not to be interpreted to exclude the presence of other features, steps or components.

As used herein the term “exemplary” means “serving as an example instance or illustration ” and should not be construed as preferred or advantageous over other configurations disclosed herein.

As used herein the terms “about” and “approximately” are meant to cover variations that may exist in the upper and lower limits of the ranges of values, such as variations in properties, parameters, and dimensions. In one non-limiting example the terms “about” and “approximately” mean plus or minus 10 percent or less.

Unless defined otherwise, all technical and scientific terms used herein are intended to have the same meaning as commonly understood by one of ordinary skill in the art. Unless otherwise indicated, such as through context, as used herein, the following terms are intended to have the following meanings:

As used herein the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein the phrase “intraoperative” refers to an action process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof. Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port.

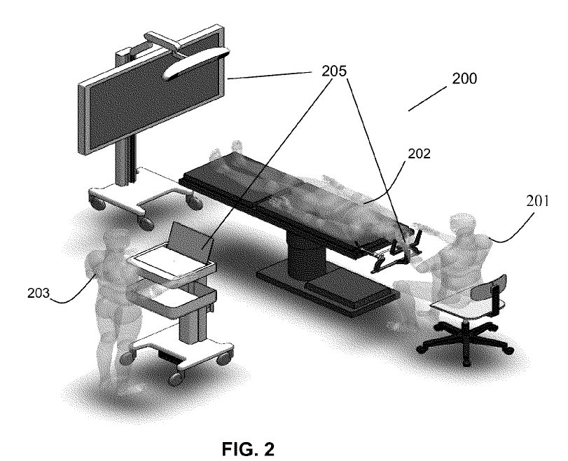

Referring to FIG. 2, an exemplary navigation system environment 200 is shown, which may be used to support navigated image-guided surgery. As shown in FIG. 2, surgeon 201 conducts a surgery on a patient 202 in an operating room (OR) environment. A medical navigation system 205 comprising an equipment tower, tracking system, displays and tracked instruments assist the surgeon 201 during his procedure. An operator 203 is also present to operate, control and provide assistance for the medical navigation system 205.

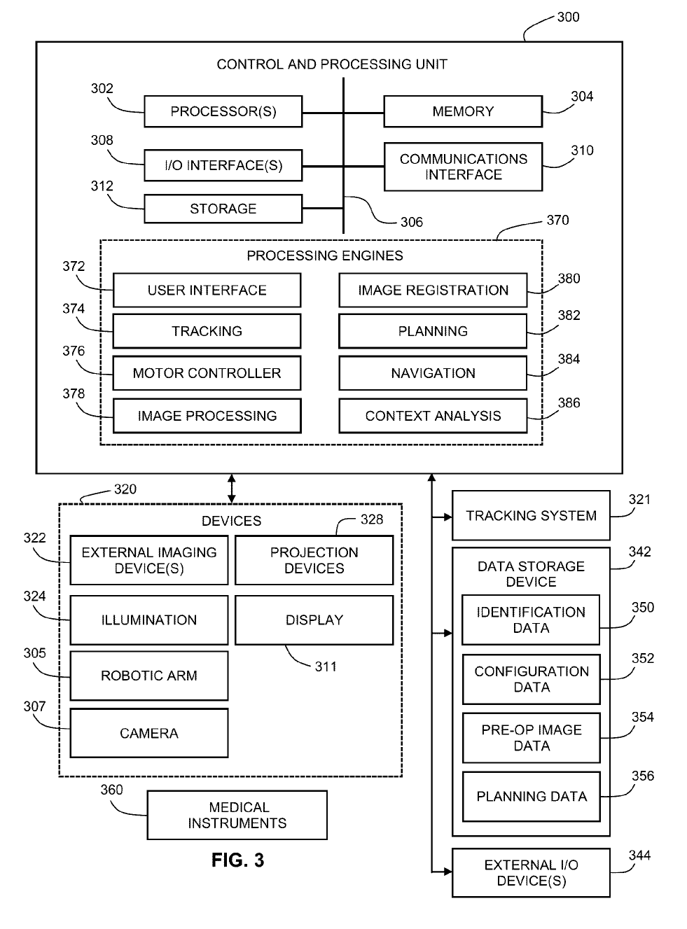

Referring to FIG. 3, a block diagram is shown illustrating a control and processing system 300 that may be used in the medical navigation system 200 shown in FIG. 3 (e.g., as part of the equipment tower). As shown in FIG. 3, in one example, control and processing system 300 may include one or more processors 302, a memory 304, a system bus 306, one or more input/output interfaces 308, a communications interface 310, and storage device 312. Control and processing system 300 may be interfaced with other external devices, such as tracking system 321, data storage 342, and external user input and output devices 344, which may include, for example, one or more of a display, keyboard, mouse, sensors attached to medical equipment, foot pedal, and microphone and speaker. Data storage 342 may be any suitable data storage device, such as a local or remote computing device (e.g. a computer, hard drive, digital media device, or server) having a database stored thereon. In the example shown in FIG. 3, data storage device 342 includes identification data 350 for identifying one or more medical instruments 360 and configuration data 352 that associates customized configuration parameters with one or more medical instruments 360. Data storage device 342 may also include preoperative image data 354 and/or medical procedure planning data 356. Although data storage device 342 is shown as a single device in FIG. 3, it will be understood that in other embodiments, data storage device 342 may be provided as multiple storage devices.

Medical instruments 360 are identifiable by control and processing unit 300. Medical instruments 360 may be connected to and controlled by control and processing unit 300, or medical instruments 360 may be operated or otherwise employed independent of control and processing unit 300. Tracking system 321 may be employed to track one or more of medical instruments 360 and spatially register the one or more tracked medical instruments to an intraoperative reference frame. For example, medical instruments 360 may include tracking spheres that may be recognizable by a tracking camera 307 and/or tracking system 321. In one example, the tracking camera 307 may be an infrared (IR) tracking camera. In another example, as sheath placed over a medical instrument 360 may be connected to and controlled by control and processing unit 300.

Control and processing unit 300 may also interface with a number of configurable devices, and may intraoperatively reconfigure one or more of such devices based on configuration parameters obtained from configuration data 352. Examples of devices 320, as shown in FIG. 3, include one or more external imaging devices 322, one or more illumination devices 324, a robotic arm 305, one or more projection devices 328, and one or more displays 205, 211.

Exemplary aspects of the disclosure can be implemented via processor(s) 302 and/or memory 304. For example, the functionalities described herein can be partially implemented via hardware logic in processor 302 and partially using the instructions stored in memory 304, as one or more processing modules or engines 370. Example processing modules include, but are not limited to, user interface engine 372, tracking module 374, motor controller 376, image processing engine 378, image registration engine 380, procedure planning engine 382, navigation engine 384, and context analysis module 386. While the example processing modules are shown separately in FIG. 3, in one example the processing modules 370 may be stored in the memory 304 and the processing modules may be collectively referred to as processing modules 370.

It is to be understood that the system is not intended to be limited to the components shown in FIG. 3. One or more components of the control and processing system 300 may be provided as an external component or device. In one example, navigation module 384 may be provided as an external navigation system that is integrated with control and processing system 300.

Some embodiments may be implemented using processor 302 without additional instructions stored in memory 304. Some embodiments may be implemented using the instructions stored in memory 304 for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which, when executed by a data processing system, causes the system to perform various methods. The executable software and data may be stored in various places including for example ROM, volatile RAM, nonvolatile memory and/or cache. Portions of this software and/or data may be stored in any one of these storage devices.

Examples of computer-readable storage media include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions may be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

At least some of the methods described herein are capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

According to one aspect of the present application, one purpose of the navigation system 205, which may include control and processing unit 300, is to provide tools to the neurosurgeon that will lead to the most informed, least damaging neurosurgical operations. In addition to removal of brain tumours and intracranial hemorrhages (ICH), the navigation system 205 can also be applied to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

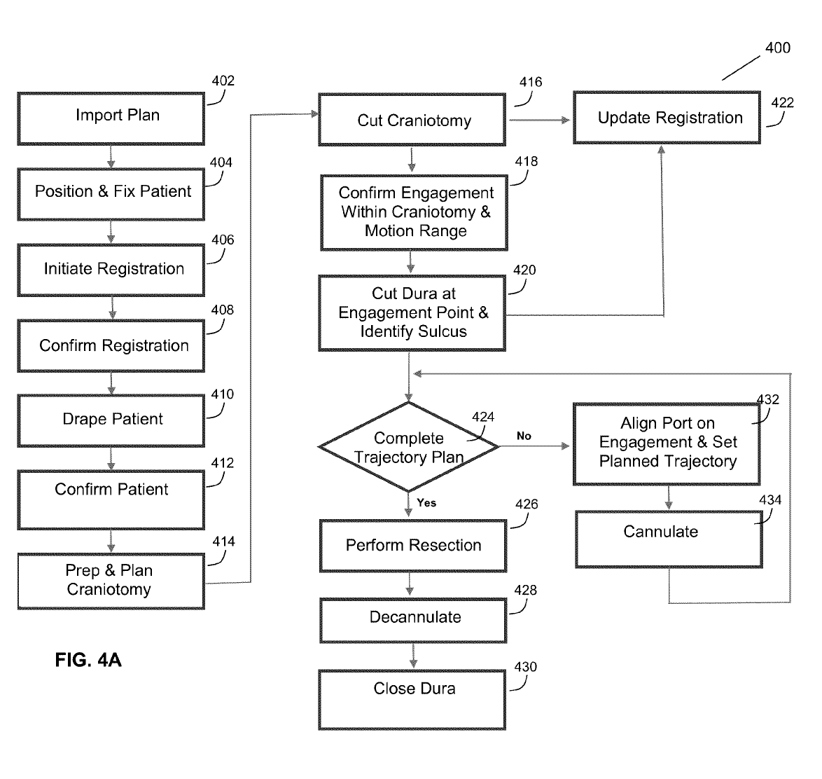

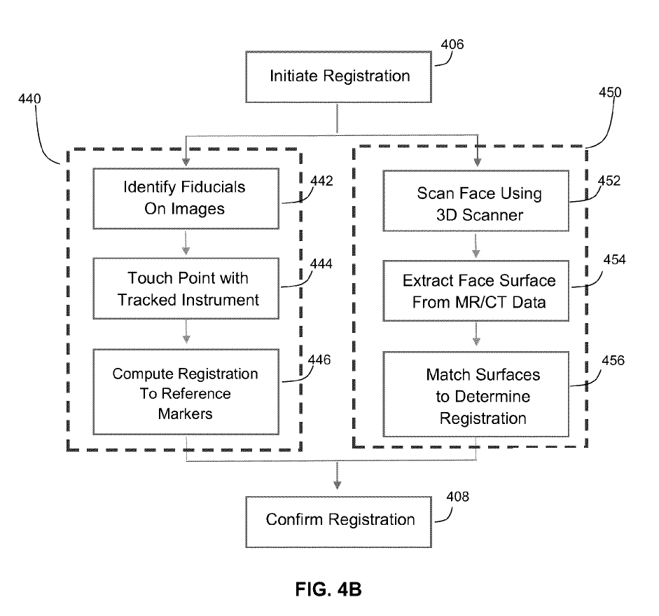

Referring to FIG. 4A, a flow chart is shown illustrating a method 400 of performing a port-based surgical procedure using a navigation system, such as the medical navigation system 200 described in relation to FIG. 2. At a first block 402, the port-based surgical plan is imported. A detailed description of the process to create and select a surgical plan is outlined in the disclosure “PLANNING NAVIGATION AND SIMULATION SYSTEMS AND METHODS FOR MINIMALLY INVASIVE THERAPY” a United States Patent Publication based on a United States patent application, which claims priority to U.S. Provisional Patent Application Ser. Nos. 61/800,155 and 61/924,993, which are both hereby incorporated by reference in their entirety.

Once the plan has been imported into the navigation system at the block 402, the patient is affixed into position using a body holding mechanism. The head position is also confirmed with the patient plan in the navigation system (block 404), which in one example may be implemented by the computer or controller forming part of the equipment tower 201.

Next, registration of the patient is initiated (block 406). The phrase registration or image registration refers to the process of transforming different sets of data into one coordinate system. Data may includes multiple photographs, data from different sensors, times, depths, or viewpoints. The process of registration is used in the present application for medical imaging in which images from different imaging modalities are co-registered. Registration is used in order to be able to compare or integrate the data obtained from these different modalities.

Those skilled in the relevant arts will appreciate that there are numerous registration techniques available and one or more of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT.

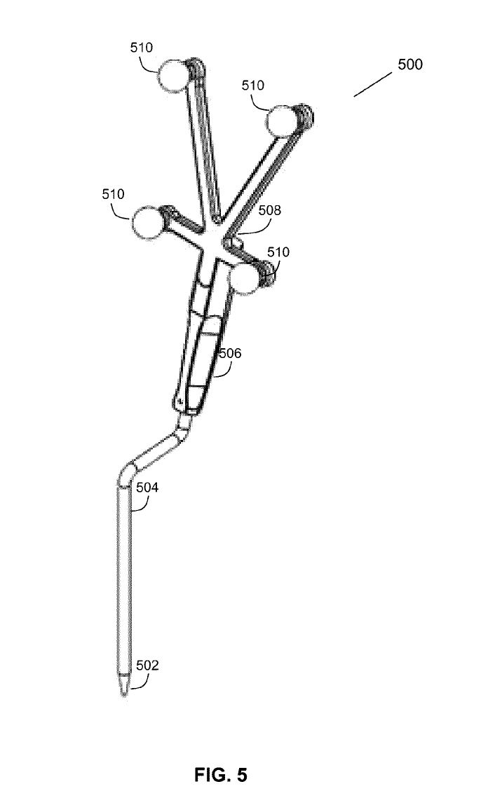

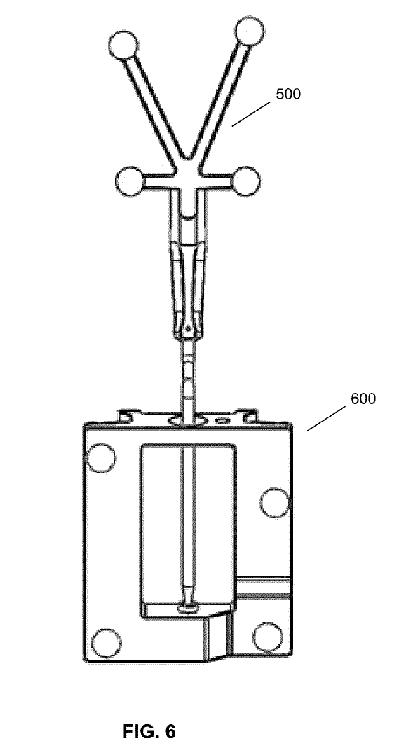

Referring now to FIG. 4B a flow chart is shown illustrating a method involved in registration block 406 as outlined in FIG. 4A, in greater detail. If the use of fiducial touch points (440) is contemplated, the method involves first identifying fiducials on images (block 442), then touching the touch points with a tracked instrument (block 444). Next, the navigation system computes the registration to reference markers (block 446). Of course, the medical navigation system 205 has to know the relationship of the tip of tracked instrument relative to the tracking markers of the tracked instrument with a high degree of accuracy for the blocks 444 and 446 to provide useful and reliable information to the medical navigation system 205. An example tracked instrument is discussed below with reference to FIG. 5 and a calibration apparatus for verifying and establishing this relationship is discussed below in connection with FIGS. 6-13.

Alternately, registration can also be completed by conducting a surface scan procedure (block 450). The block 450 is presented to show an alternative approach, but may not typically be used when using a fiducial pointer. First, the face is scanned using a 3D scanner (block 452). Next, the face surface is extracted from MR/CT data (block 454). Finally, surfaces are matched to determine registration data points (block 456).

Upon completion of either the fiducial touch points (440) or surface scan (450) procedures, the data extracted is computed and used to confirm registration at block 408, shown in FIG. 4A.

Referring back to FIG. 4A, once registration is confirmed (block 408), the patient is draped (block 410). Typically, draping involves covering the patient and surrounding areas with a sterile barrier to create and maintain a sterile field during the surgical procedure. The purpose of draping is to eliminate the passage of microorganisms (e.g., bacteria) between non-sterile and sterile areas. At this point, conventional navigation systems require that the non-sterile patient reference is replaced with a sterile patient reference of identical geometry location and orientation. Numerous mechanical methods may be used to minimize the displacement of the new sterile patient reference relative to the non-sterile one that was used for registration but it is inevitable that some error will exist. This error directly translates into registration error between the surgical field and pre-surgical images. In fact, the further away points of interest are from the patient reference, the worse the error will be.

Upon completion of draping (block 410), the patient engagement points are confirmed (block 412) and then the craniotomy is prepared and planned (block 414).

Upon completion of the preparation and planning of the craniotomy (block 414), the craniotomy is cut and a bone flap is temporarily removed from the skull to access the brain (block 416). Registration data is updated with the navigation system at this point (block 422).

Next, the engagement within craniotomy and the motion range are confirmed (block 418). Next, the procedure advances to cutting the dura at the engagement points and identifying the sulcus (block 420).

Thereafter, the cannulation process is initiated (block 424). Cannulation involves inserting a port into the brain, typically along a sulci path as identified at 420, along a trajectory plan. Cannulation is typically an iterative process that involves repeating the steps of aligning the port on engagement and setting the planned trajectory (block 432) and then cannulating to the target depth (block 434) until the complete trajectory plan is executed (block 424.

Once cannulation is complete, the surgeon then performs resection (block 426) to remove part of the brain and/or tumor of interest. The surgeon then decannulates (block 428) by removing the port and any tracking instruments from the brain. Finally, the surgeon closes the dura and completes the craniotomy (block 430). Some aspects of FIG. 4A are specific to port-based surgery, such portions of blocks 428, 420, and 434, but the appropriate portions of these blocks may be skipped or suitably modified when performing non-port based surgery.

When performing a surgical procedure using a medical navigation system 200, as outlined in connection with FIGS. 4A and 4B, the medical navigation system 200 must acquire and maintain a reference of the location of the tools in use as well as the patient in three dimensional (3D) space. In other words, during a navigated neurosurgery, there needs to be a tracked reference frame that is fixed relative to the patient's skull. During the registration phase of a navigated neurosurgery (e.g., the step 406 shown in FIGS. 4A and 4B), a transformation is calculated that maps the frame of reference of preoperative MRI or CT imagery to the physical space of the surgery, specifically the patient's head. This may be accomplished by the navigation system 200 tracking locations of markers fixed to the patient's head relative to the static patient reference frame. The patient reference frame is typically rigidly attached to the head fixation device, such as a Mayfield clamp. Registration is typically performed before the sterile field has been established (e.g., the step 410 shown in FIG. 4A).

Referring to FIG. 5, a perspective drawing is shown illustrating an exemplary tracked instrument to which aspects of the present application may be applied. In the example shown in FIG. 5, an exemplary pointer tool 500 is illustrated. In one example, the pointer tool 500 may be a fiducial pointer tool. The pointer tool 500 may be considered an exemplary instrument for navigation having either a straight or slightly blunt tip 502. The slenderness of the tip 502 on a handheld pointer allows for precise positioning and localization of external fiducial markers on the patient. The tip 502 is located at the end of a shaft 504. The shaft 504 is connected to a handle portion 506. The handle portion 506 connects to a frame 508 that supports a number of tracking markers 510. In the example shown in FIG. 5, the pointer tool 500 has four passive reflective tracking spheres, but any suitable number of tracking markers 510 may be used and any suitable type of tracking marker 510 may be used, including an active infrared (IR) marker, an active light emitting diode (LED), and a graphical pattern. It is important that medical navigation system 200 known the dimensions of the pointer tool 500 such that the precise position of the tip 502 relative to the tracking markers 510 (e.g., that the medical navigation system 200 sees the tracking makers 510 using the camera 307) is known. If the shaft 504 becomes slightly bent or deformed, the relationship of the tip 502 relative to the tracking markers 510 may change, which can cause inaccuracies in medical procedures using the medical navigation system 200, which is a serious problem.

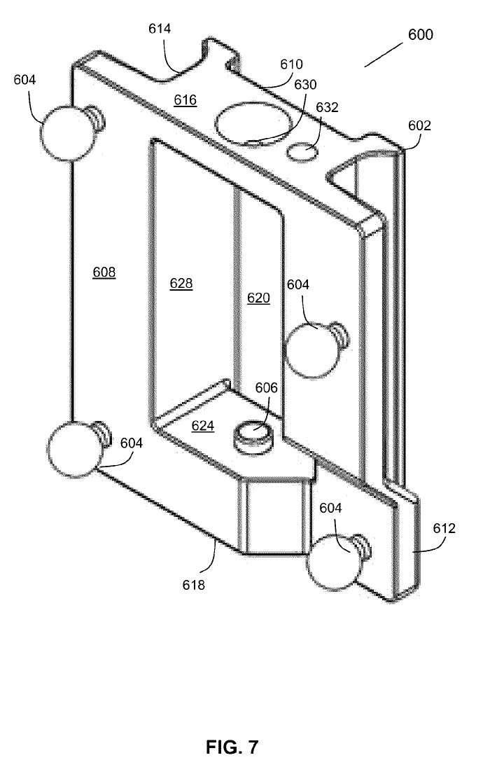

Referring to FIG. 6, a perspective drawing is shown illustrating the tracked instrument 500 shown in FIG. 5 inserted into a calibration apparatus 600 according to one aspect of the present description. Calibration apparatus 600 is now discussed in detail in connection with FIGS. 7-13, below.

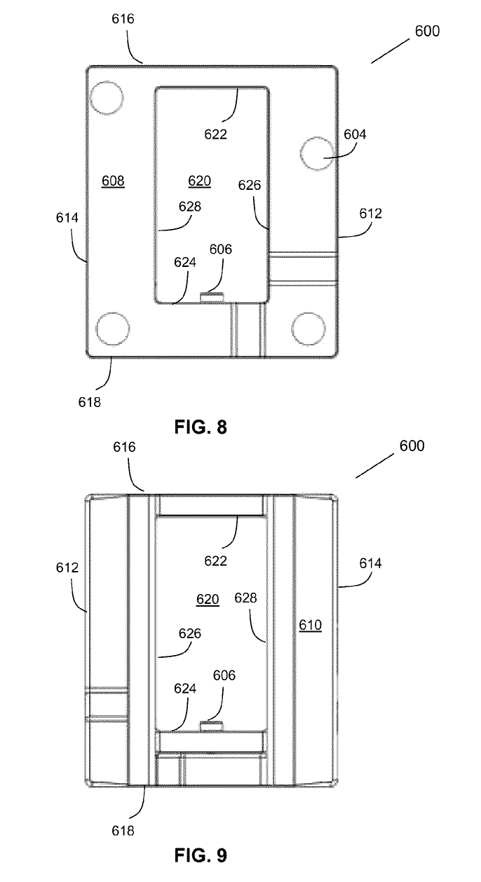

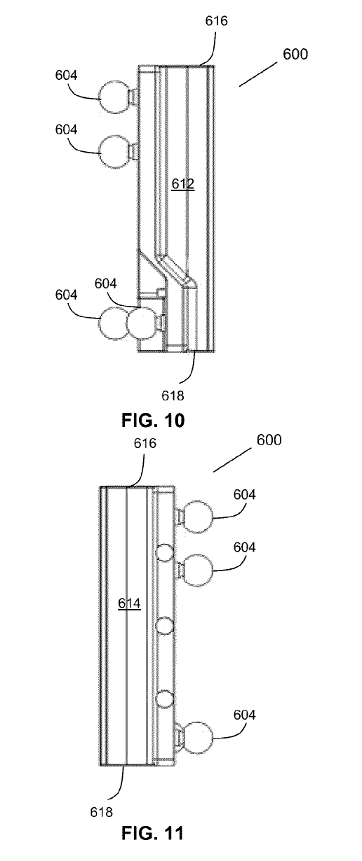

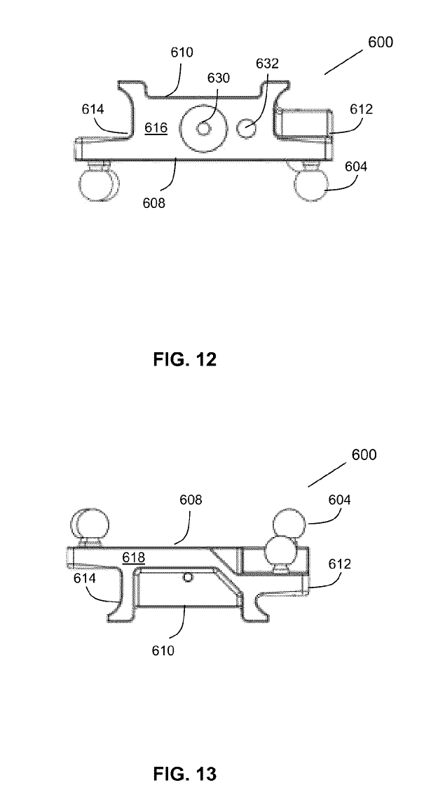

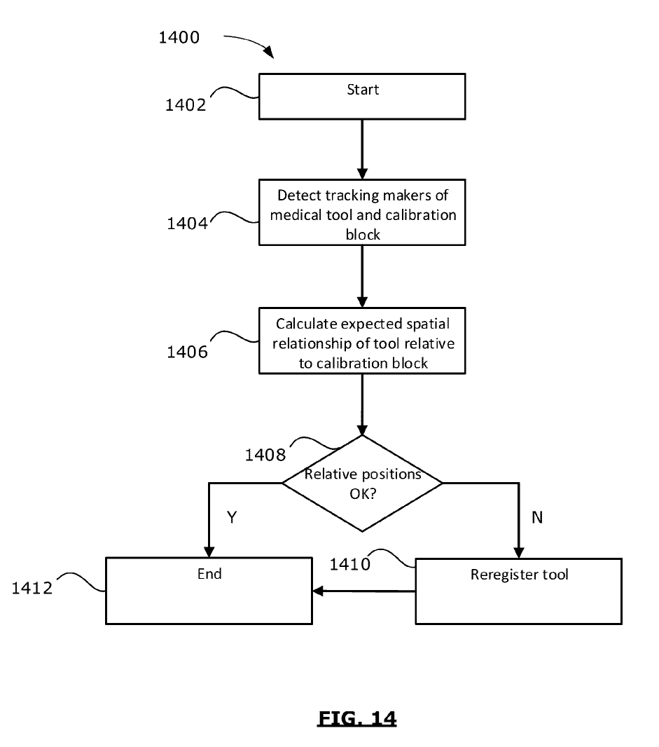

Referring to FIG. 7, a perspective drawing is shown illustrating the calibration apparatus 600 in isolation that was introduced in FIG. 6. For simplicity, calibration apparatus 600 will be referred to throughout as a calibration block 600, although the apparatus need not necessary take the form of a block. FIG. 8 is a front view of the calibration block 600. FIG. 9 is a rear view of the calibration block 600. FIG. 10 is a right side view of the calibration block 600. FIG. 11 is a left side view of the calibration block 600. FIG. 12 is a top view of the calibration block 600. FIG. 13 is bottom view of the calibration block 600. FIGS. 7-13 are now discussed concurrently.

The calibration block 600 may be used to calibrate a medical tool having a tool tracking marker, such as the pointer tool 500 having the tracking markers 510. The medical tool and the calibration block 600 are typically used in conjunction with a medical navigation system, such as the medical navigation system 200 that includes the control and processing unit 300. The calibration block 600 includes a frame 602, at least one frame tracking marker 604 attached to the frame 602, and a reference point 606 formed on the frame 602. In one example, the reference point may be a divot that is of an appropriate shape for securely receiving the tip 502 of the pointer tool 500. For the purposes of example, the reference point 606 will be referred to throughout as a divot 606, however any reference point or surface may be used to meet the design criteria of a particular application. The divot 606 may provide a known spatial reference point relative to the frame tracking markers 604. For example, the medical navigation system 200 may have data saved therein (e.g., in data storage device 342) so that the medical navigation system 200 knows the position in space of a floor of the divot 606 relative to the tracking makers 604 to a high degree of accuracy. In one example, a high degree of accuracy may refer to a tolerance of 0.08 mm, but any suitable tolerance may be used according to the design criteria of a particular application.

In the example shown in FIGS. 7-13, the calibration block 600 has has four passive reflective tracking spheres, but any suitable number of tracking markers 604 may be used and any suitable type of tracking marker 604 may be used according to the design criteria of a particular application, including an active infrared (IR) marker, an active light emitting diode (LED), and a graphical pattern. When passive reflective tracking spheres are used as the tracking makers 604, typically at least three tracking markers 604 will be attached to a same side of the frame 602. Likewise, when a medical instrument such as the pointer tool 500 having passive reflective tracking spheres is used in conjunction with the calibration block 600, the medical instrument will typically have at least three tracking markers 510 attached thereto.

The tip 502 of the medical tool 500 is insertable into the divot 606 to abut against a floor of the divot 606 for validation of the medical tool 500 dimensions by the medical navigation system 200. Since the medical navigation system 200 knows the precise dimensions of the calibration block 600 (e.g., saved in data storage device 342), and the medical navigation system 200 knows the precise dimensions of the medical tool such as the pointer tool 500 that was previously registered. A deformed medical tool is re-registerable with the medical navigation system 200 such that the medical navigation system 200 learns the new dimensions of the deformed tool. In other words, when the pointer tool 500 is placed in the calibration block 600, as shown in FIG. 6, the position of the tip 502 of the pointer tool 500 relative to the tracking makers 510 that the medical navigation system 200 is seeing (e.g., using the camera 307) is known. Likewise, the position of the floor of the divot 606 relative to the tracking makers 604 that the medical navigation system 200 is seeing (e.g., using the camera 307) is known. The medical navigation system 200 has enough information to calculate to a designed tolerance the expected location of the tracking makers 604 relative to the tracking makers 510. In one example, the designed tolerance may be a tolerance of 1.0 mm, but any suitable tolerance may be used according to the design criteria of a particular application. When this expected location differs, in the vast majority of cases and assuming the structural integrity of the calibration block 600, the cause will be a bent or deformed shaft 504. When this occurs, the medical navigation system 200 may simply learn the new dimensions of the deformed or bent medical tool, such as the pointer tool 500 (e.g., re-registration) and save this information, for example in the data storage device 342. FIG. 14, discussed below, outlines a method for verifying and, if necessary, reregistering a medical tool.

Returning to FIGS. 7-13, the calibration block 600 has a front side 608, a back side 610, a right side 612, a left side 614, a top side 616, and a bottom side 618. The calibration block 600 exists in three dimensional space having an X-axis, a Y-axis, and a Z-axis. In one example where passive reflective tracking spheres are used, at least one of the frame tracking markers 604 differs in position in the X direction from the remaining tracking makers, at least one of the at least three frame tracking markers 604 differs in position in the Y direction from the remaining tracking makers, and at least one of the at least three frame tracking markers 604 differs in position in the Z direction from the remaining tracking makers. This feature may provide the medical navigation system 200 with a better degree of accuracy to detect the position of the calibration block 600 in 3D space.

The calibration block 600 further has a cavity 620 between the right side 612 and the left side 614 of the frame 602 and between the top side 616 and the bottom side 618 of the frame 602. The cavity may have a top side 622, a bottom side 624, a right side 626, and a left side 628. In one example, the divot 606 may be positioned on the bottom side 624 of the cavity 620.

The calibration block 600 may further have a retaining orifice 630 positioned on a top side 616 of the frame 602 and extending through to the top side 622 of the cavity 620. The retaining orifice 630 may receive the medical tool such as the pointer tool 500 as the tip 502 of the tool 500 is positioned in the divot 606. The retaining orifice 630 may serve to hold the tool 500 in an upright position when the tip 502 of the tool 500 rests in the divot 606.

The calibration block 600 may further have a second reference point 632, which in one example may be a second divot 632, formed on the frame 602 for further validating the medical tool 500 dimensions by the medical navigation system 200. The second divot 632 may not have an associated retaining orifice 630, which allows the tool 500 to move around in free space as a user of the tool 500 holds the tool 500 with the tip 502 firmly abutted against the floor of the divot 632. This may allow the medical navigation system 200 to perform an even increased level of analysis on the tool 500 as it moves around in 3D space with the tip 502 firmly planted in the divot 632, which allows the medical navigation system 200 to detect multiple positions of the tracking markers 604 and generate many different equations for the spatial position of the tip 502 relative to the makers 604, allowing for an error minimization method or algorithm to be executed.

In one example, the calibration block 600 may be made of stainless steel, aluminum or any other suitable metal. Alternatively, the calibration block 600 may be constructed of plastic, a polymer or other synthetic material of a suitable weight and rigidity. The calibration block 600 may be constructed using yet to be developed or known manufacturing techniques such as injected molding, machine tooling and 3D printing. While some examples of suitable materials and manufacturing techniques are provided for the calibration block 600, any suitable material and manufacturing technique may be used according to the design criteria of a particular application.

Referring now to FIG. 14, a flow chart is shown illustrating a method 1400 for verifying and reregistering a medical tool according to one aspect of the present description. The method 1400 may be executed by the medical navigation system 200 either as a precursor to the method 400 shown in FIG. 4 or during the method 400 shown in FIG. 4 if it becomes apparent to the surgeon performing the medical procedure that the dimensions of the medical tool 500 may have changed.

The method 1400 begins at a block 1402, for example by the surgeon 201 or operator 203 executing the tool verification and reregistration process by proving appropriate input to the control and processing unit 300, for example by using the external I/O devices 344. At this point, the surgeon 201 may ensure that the medical tool 500 is placed in the calibration block 600 and that both are clearly visible by the appropriate sensors used by the control and processing unit 300 to see the tool and the calibration block, such as the camera 307 in the case of optical tracking markers.

Next, at a block 1404, the tracking makers of the medical tool 500 and the calibration block 600 are detected by the control and processing unit 300. In the example of passive reflective tracking markers, the camera 307 may provide input to the processor 300, which detects the locations of the tracking makers 510 and 604.

Next at a block 1406, the spatial relationship of the tracking makers 510 relative to the tracking makers 604 is calculated by the control and processing unit 300. Since the control and processing unit 300 knows the expected dimensions of the medical tool 500 (e.g., the location of the tip 502 relative to the tracking makers 510) and knows the dimensions of the calibration block 600 (e.g., the location of the floor of the reference point 606 relative to the tracking makers 604), the control and processing unit 300 can calculated the expected acceptable range of locations of the tracking makers 604 relative to the tracking makers 510.

At a block 1408, the relative positions of the tracking makers 604 to the tracking makers 510 are assessed. If it is determined that the dimensions of the medical tool 500 have changed, such as from a bending or deformation of the shaft 504, the control and processing units 300 may relearn the dimensions of the medical tool 500 and reregister the medical tool 500 at a block 1410. The method 1400 then ends at the block 1412. If it is determined at the block 1408 that the dimensions of the medical tool 500 have not changed beyond a threshold, then the dimensions of the medical tool 500 have been verified and the method 1400 ends at the block 1412 without reregistering the medical tool 500. In one example, the threshold may be between 0.3 mm and 1 mm, depending on the design criteria of the particular application, however the method 1400 may be used with any suitable tolerance.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

We claim:

1. A calibration apparatus for calibrating a medical tool having a tool tracking marker, the medical tool and the calibration apparatus for use with a medical navigation system, the calibration apparatus comprising:
a frame;
a frame tracking marker attached to the frame; and
a reference point formed on the frame, the reference point providing a known spatial reference point relative to the frame tracking marker.

2. The calibration apparatus according to claim 1, wherein the frame tracking marker includes at least one of a passive reflective tracking sphere, an active infrared (IR) marker, an active light emitting diode (LED), and a graphical pattern.

3. The calibration apparatus according to claim 2, wherein the frame has at least three tracking markers attached to a same side of the frame.

4. The calibration apparatus according to claim 3, wherein the reference point includes a divot and the medical tool has at least three tracking markers attached thereto and a tip of the medical tool is insertable into the divot to abut against a floor of the divot for validation of the medical tool dimensions by the medical navigation system.

5. The calibration apparatus according to claim 4, wherein the frame and the medical tool each have at least four tracking markers attached thereto and a deformed medical tool is re-registerable with the medical navigation system such that the medical navigation system learns the new dimensions of the deformed tool.

6. The calibration apparatus according to claim 1, wherein the apparatus has a front side, a back side, a right side, a left side, a top side, and a bottom side, and the apparatus has at least three frame tracking markers attached to a same side of the apparatus.

7. The calibration apparatus according to claim 6, wherein the calibration apparatus exists in three dimensional space having an X-axis, a Y-axis, and a Z-axis, and at least one of the at least three frame tracking markers differs in position in the X direction from the remaining tracking makers, at least one of the at least three frame tracking markers differs in position in the Y direction from the remaining tracking makers, and at least one of the at least three frame tracking markers differs in position in the Z direction from the remaining tracking makers.

8. The calibration apparatus according to claim 6, wherein the apparatus includes a cavity between the right side and the left side of the frame and between the top side and the bottom side of the frame, the cavity having a top side, a bottom side, a right side, and a left side, the reference point being positioned on the bottom side of the cavity.

9. The calibration apparatus according to claim 8, further including a retaining orifice positioned on a top side of the frame and extending through to the top side of the cavity, the retaining orifice for receiving the medical tool as the tip of the medical tool is positioned in the reference point, the retaining orifice holding the medical tool in an upright position when the tip of the medical tool rests in the reference point.

10. The calibration apparatus according to claim 4, further including a second reference point formed on the frame for further validating the medical tool dimensions by the medical navigation system.

11. A medical navigation system, comprising:
a medical tool having a tool tracking marker;
a calibration apparatus for calibrating the medical tool, the calibration apparatus having:
a frame;
a frame tracking marker attached to the frame; and
a reference point formed on the frame, the reference point providing a known spatial reference point relative to the frame tracking marker; and
a controller having a sensor for detecting the tracking makers, the sensor providing a signal to the controller indicating positions of the tracking markers in space.

12. The medical navigation system according to claim 11, wherein the frame tracking marker and the tool tracking marker include at least one of a passive reflective tracking sphere, an active infrared (IR) marker, an active light emitting diode (LED), and a graphical pattern.

13. The medical navigation system according to claim 12, wherein the reference point includes a divot, the frame has at least three tracking markers attached to a same side of the frame and the medical tool has at least three tracking markers attached thereto and a tip of the medical tool is insertable into the divot to abut against a floor of the divot for validation of the medical tool dimensions by the medical navigation system based on signals provided by the sensor.

14. The medical navigation system according to claim 13, wherein the frame and the medical tool each have at least four tracking markers attached thereto and a deformed medical tool is re-registerable with the medical navigation system such that the medical navigation system learns the new dimensions of the deformed tool.

15. The medical navigation system according to claim 11, wherein the calibration apparatus has a front side, a back side, a right side, a left side, a top side, and a bottom side, and the apparatus has at least three frame tracking markers attached to a same side of the apparatus, wherein the calibration apparatus exists in three dimensional space having an X-axis, a Y-axis, and a Z-axis, and at least one of the at least three frame tracking markers differs in position in the X direction from the remaining frame tracking makers, at least one of the at least three frame tracking markers differs in position in the Y direction from the remaining frame tracking makers, and at least one of the at least three frame tracking markers differs in position in the Z direction from the remaining frame tracking makers.

16. The medical navigation system according to claim 15, wherein the calibration apparatus includes a cavity between the right side and the left side of the frame and between the top side and the bottom side of the frame, the cavity having a top side, a bottom side, a right side, and a left side, the reference point being positioned on the bottom side of the cavity.

17. The medical navigation system according to claim 16, further including a retaining orifice positioned on a top side of the frame and extending through to the top side of the cavity, the retaining orifice for receiving the medical tool as the tip of the medical tool is positioned in the reference point, the retaining orifice holding the medical tool in an upright position when the tip of the medical tool rests in the reference point.

18. The medical navigation system according to claim 11, wherein the reference point includes a divot, the calibration apparatus further including a second divot formed on the frame for further validating the medical tool dimensions by the medical navigation system.

19. A method of verifying dimensions of a medical tool having an attached tool tracking maker using a calibration apparatus having a frame, a frame tracking marker attached to the frame and a reference point formed on the frame, the reference point providing a known spatial reference point relative to the frame tracking marker, the method comprising:
detecting the tool tracking maker and the frame tracking maker;
calculating the expected spatial relationship of the tool tracking maker relative to the frame tracking maker; and
reregistering the tool when the dimensions of the medical tool have changed beyond a threshold.
